SEQUENCE LISTING (1 sequence)

SEQ ID NO: 1 - DNA, 1217 bp, Artificial Sequence
  Description of Artificial Sequence Synthetic polynucleotide
  misc_feature (1)..(143): Homologous to T7
  misc_feature (159)..(176): T7 promoter
  misc_feature (177)..(211): Ribosome binding site
  misc_feature (212)..(277): pelB leader sequence
  misc_feature (278)..(790): Modified luciferase
  misc_feature (791)..(1134): Cellulose binding motif
  misc_feature (1135)..(1217): Homologous to T7
gtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattcgagctccgtctaatacgactcactatagctaaacattaatcatttaaaataaggaggtaaagcatgaaatatcttctgcctacggctgccacgggtttgttactgcttgcagctcagccagcggtcgccatggtattcacactggaggattttgtcggtgactggcgccagactgctggatataatcttgatcaagtgctggagcaaggaggcgtctcaagccttttccagaatttaggtgttagcgtcacaccgattcaacgtatcgtgctgagtggggagaacggcttaaaaatcgacatccacgtcatcattccatatgaagggttgtcaggggatcagatgggtcagattgaaaagatttttaaggttgtctacccagtagacgaccatcacttcaaggttattttacactacggtacattagtaattgacggcgtgactcctaacatgattgactattttggacgcccgtatgaggggattgcagtgttcgacggcaagaagatcacagttacggggactctgtggaatgggaataaaattatcgacgagcgtctgattaaccccgatggctctctgttgttccgtgtcactattaacggtgtcacgggctggcgcctttgtgaacgcattttagcaggctcgagcggccctacgtcaggtccggccggttgccaagttttatggggggtcaaccagtggaacacaggctttacggcgaacgttactgtcaagaacacaagctccgctcctgtggatggttggacactgaccttttctttcccctcaggtcagcaagtgacacaggcgtggagttctacggttacacaatctggttctgctgttactgtccgtaacgcgccctggaatggaagcatcccagcgggcgggaccgcacagtttggcttcaatggctctcatacagggacaaacgcagcaccaacagcattttccttgaatggaaccccttgcactgtcggataagcttgcggccgcactcgagtaactagttaaccccttggggcctctaaacgggtcttgaggggttttttgctgaaaggaggaa

(19) United States
(12) Patent Application Publication
(10) Pub. No.: US 2021/0239694 A1
(43) Pub. Date: Aug. 5, 2021

(54) PHAGE CONSTRUCTS FOR DETECTING BACTERIA IN A FLUID, MICROFLUIDIC DEVICES FOR USE WITH CONSTRUCTS, AND RELATED METHODS

(71) Applicants: CORNELL UNIVERSITY, Ithaca, NY (US); TOKITAE LLC, Bellevue, WA (US)

(72) Inventors: Luis F. ALONZO, Tacoma, WA (US); Spencer GARING, Seattle, WA (US); Troy HINKLEY, Ithaca, NY (US); Anne-Laure M. LE NY, Issaquah, WA (US); Damian MADAN, Issaquah, WA (US); Kevin Paul Flood NICHOLS, Issaquah, WA (US); Sam Rasmussen NUGEN, Ithaca, NY (US)

(21) Appl. No.: 17/049,280

(22) PCT Filed: Apr. 19, 2019
(86) PCT No.: PCT/US2019/028287

§ 371 Date: Oct. 20, 2020

Related U.S. Application Data
(63) Continuation of application No. 15/958,931, filed on Apr. 20, 2018, now abandoned.

(51) Int. Cl. G01N 33/569; C12N 15/73; G01N 33/543; C12Q 1/10; C12Q 1/66; C12N 7/02

(52) U.S. Cl. CPC G01N 33/56911; C12N 15/73; G01N 33/54366; G01N 2333/245; C12Q 1/66; C12N 7/025; G01N 2333/30; C12Q 1/10

(57) ABSTRACT

Generally, this disclosure relates to expression constructs that encode a reporter enzyme-affinity binding tag fusion protein that is produced after the construct is inserted into bacteriophage and the bacteriophage infects bacteria. In some embodiments, the fusion protein is captured and produces a detectable signal. Signal intensity may correlate with the number of bacterial cells in a fluid sample. Methods of detecting bacteria using the expression constructs, and microfluidic devices for detecting bacteria using the expression constructs are also disclosed.

Any application(s) claimed for priority are incorporated by reference, to the extent such subject matter is not inconsistent herewith.

### SEQUENCE LISTING

The instant application contains a Sequence Listing, which has been submitted electronically in ASCII format and is hereby incorporated by reference in its entirety.

### BACKGROUND

Detection of bacterial pathogens and/or their indicators in water helps ensure such water is safe to drink or to apply to foods that will be consumed. In the United States, regulatory agencies have set a limit of zero colony forming units of generic E. coli per 100 mL of drinking water or postharvest produce rinse water. U.S. Environmental Protection Agency Method 1603 is an approved drinking water assay that quantifies generic E. coli with an assay time of 24 hours. This timeframe may be too long to prevent individuals from becoming infected. Therefore, developers and users of water quality tests continue to seek improvements to tests and methods for rapid, sensitive, and accurate detection of a minimum of 1 CFU of viable target bacteria per 100 mL of water sample, as well as rapid, sensitive, and accurate quantification of viable target bacteria over a broad range of concentrations.

### SUMMARY

Generally, the present disclosure relates to expression constructs that encode a reporter enzyme-affinity binding tag fusion protein. The fusion protein is produced after the constructs are inserted into bacteriophage and the bacteriophage infects bacteria. In some embodiments, the bacteria is captured separately from capturing the fusion protein. The fusion protein produces a detectable signal that may indicate the presence or number of bacteria. The present disclosure also relates to methods of detecting bacteria using the expression constructs and to microfluidic devices for detecting bacteria using the expression constructs.

An embodiment includes a device for concentrating bacteria and bacterial products. The device includes a first inlet fluidly connected to a first immobilization region. The first immobilization region is constructed of a material capable of concentrating the bacteria. The device includes a second and third inlet, each fluidly connected to the first immobilization region. The device also includes a fourth inlet fluidly connected to a second immobilization region. The second immobilization region is constructed of a material capable of concentrating bacterial products. The material of the second immobilization region is different from the material of the first immobilization region.

An embodiment includes a method of detecting bacteria including providing a sample that has bacteria. The method includes isolating the bacteria. The method includes incubating the bacteria for a first incubation period. The method includes adding a bacteriophage to the bacteria. The bacteriophage includes an expression construct that encodes a fusion protein. The method also includes incubating the bacteria for a second incubation period sufficient for the bacteriophage to infect the bacteria and the bacteria to express the fusion protein. The method includes capturing the fusion protein. The method includes detecting the bacteria by detecting the fusion protein.

An embodiment includes a method of immobilizing bacterial products for detection. The method includes exposing a bacteriophage including an expression construct to bacteria. The expression construct includes a reporter enzyme and a protein affinity tag. The method also includes incubating the bacteriophage and the bacteria for an incubation time sufficient for the bacteriophage to infect the bacteria and the bacteria to produce the reporter enzyme and the protein affinity tag as a fusion protein. The method includes immobilizing the fusion protein by binding the protein affinity tag to a complementary substrate. The method also includes detecting the bacteria by detecting the reporter enzyme of the fusion protein.

An embodiment includes an expression construct that includes a phage-specific promoter. The expression construct also includes a reporter enzyme gene operably linked to the promoter. The reporter enzyme gene may include alkaline phosphatase, β-galactosidase, β-glucuronidase, green fluorescent protein, luciferase, neuraminidase, or a derivative of any of the foregoing. The expression construct also includes at least a portion of a protein affinity tag gene downstream of the reporter enzyme gene and operably linked to the promoter. The protein affinity tag gene may include a carbohydrate binding module, a chitin binding protein, glutathione-S-transferase, a His tag, a maltose binding protein, or a Strep-tag.

An embodiment includes a polynucleotide encoding a detectable fusion protein. The polynucleotide includes the nucleotide sequence of SEQ ID NO: 1.

Features from any of the disclosed embodiments may be used in combination with one another, without limitation. In addition, other features and advantages of the present disclosure will become apparent to those of ordinary skill in the art through consideration of the following detailed description and the accompanying drawings.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is the nucleotide sequence (SEQ ID NO: 1) of a portion of a luciferase-carbohydrate binding module expression construct according to an embodiment;

FIGS. 2A-2E are schematics of native (2A and 2C) and engineered (2B and 2D) nucleic acids and proteins (2E) and associated phages according to several embodiments;

FIG. 3 is a flow chart of a method for capturing bacterial products for detection, according to an embodiment;

FIG. 4 is a flow chart of a method for detecting bacteria, according to an embodiment;

FIG. 5 is a flow chart of a method for detecting bacteria, according to an embodiment;

FIG. 6 is a schematic of a microfluidic device, according to an embodiment;

FIG. 7 is a schematic of a bacterial detection method according to an embodiment;

FIG. 8 are photographs of detectable signals from reporter constructs, according to embodiments, used in bacterial detection assays;

FIG. 9 are photographs of detectable signals from bacterial detection methods according to embodiments; and

FIG. 10 is a graphical comparison of EPA Method #1603 for bacterial detection with phage-based detection methods according to embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Generally, the present disclosure relates to expression constructs, methods of detecting bacteria or mycobacteria (either or both are referred to as “bacteria” hereafter) in a fluid sample using the expression constructs, and microfluidic devices for detecting bacteria in a fluid sample using the expression constructs. In some embodiments, the expression constructs include DNA sequences that encode a reporter enzyme and are insertable into a lytic or lysogenic bacteriophage. After infection by the engineered bacteriophage, bacteria in a fluid sample may express the reporter enzyme. The natural phage lytic cycle may result in bacterial cell lysis and release of the enzyme. In some embodiments, the enzyme is fused to a protein affinity tag that permits capture and concentration of the released enzyme on a target substrate. Addition of a reporter enzyme substrate compound may produce a detectable signal. Signal intensity may correlate with the number of bacterial cells in the fluid sample. Devices and methods disclosed herein may be used to detect low levels of viable bacterial cells in fluids such as drinking water.

### Expression Constructs

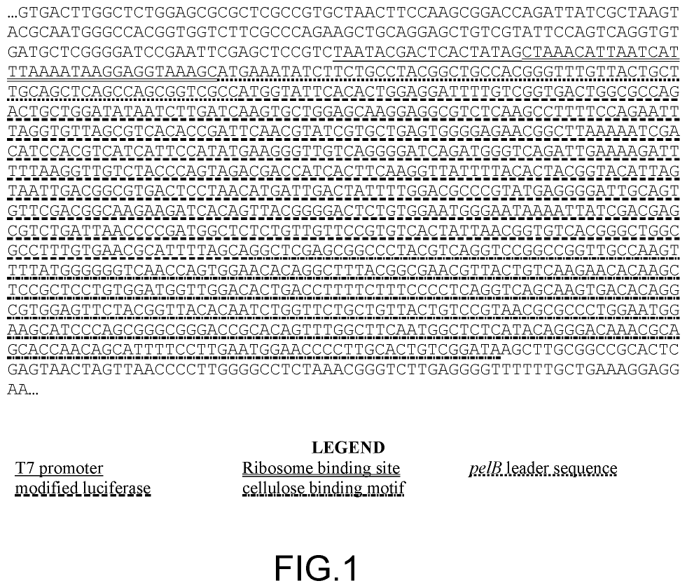

Expression constructs disclosed herein are designed and constructed to be introduced into bacteriophages and expressed when the bacteriophages have infected bacteria and commandeered bacterial cellular machinery. Expression constructs include at least a portion of each of a reporter enzyme gene and a protein affinity tag gene. Expression constructs may also include one or more of a promoter, ribosome binding site, leader sequence, and nucleotides homologous to phage DNA. FIG. 1 shows the nucleotide sequence of a portion of an expression construct according to an embodiment.

In some embodiments, the reporter enzyme and protein affinity tag may be genetically linked such that transcription and translation of the genes produces a fusion protein of the reporter enzyme linked to the protein affinity tag. The tag may be upstream (5′) or downstream (3′) of the enzyme. In the embodiment shown in FIG. 1, the tag is immediately downstream of the enzyme.

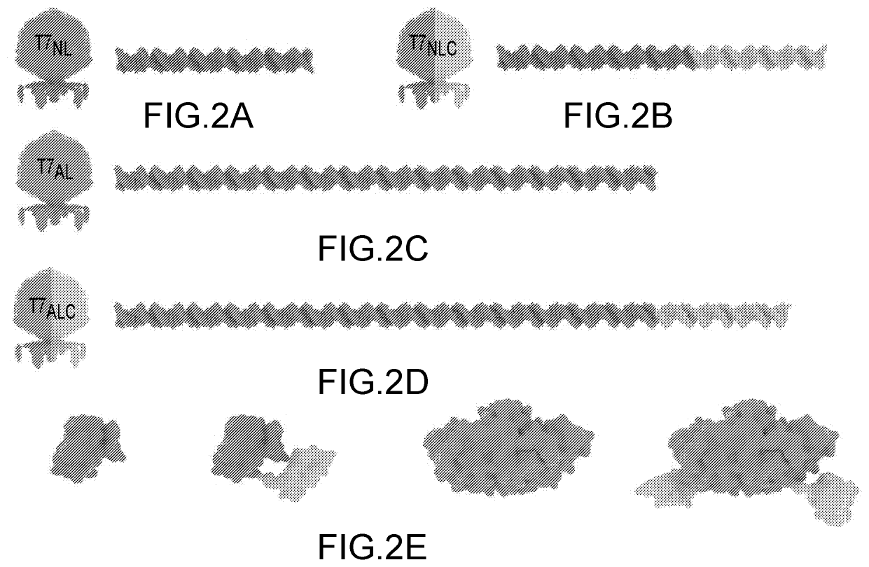

The reporter enzyme gene product may produce a detectable signal, which may enable detection of bacteria that were infected by bacteriophage engineered with the expression construct. The reporter enzyme gene product may include, but is not limited to, alkaline phosphatase, β-galactosidase, β-glucuronidase, green fluorescent protein, luciferase, neuraminidase, or a derivative of any of the foregoing. In an embodiment, the reporter enzyme gene may be a bacterial alkaline phosphatase gene (alp) having two amino acid substitutions (D153G/D330N). The modified phosphatase enzyme may be suitable for use with numerous substrate compounds. In some embodiments, enzymatic processing of a substrate compound may produce a colored product. In an embodiment, the compound is 5-bromo-4-chloro-3-indolyl-phosphatase, p-toluidine salt (BCIP). The modified phosphatase enzyme may have increased activity compared to the native enzyme. The activity may be increased by at least two orders of magnitude (Muller et al, 2001, Chembiochem 2(7-8), 517-523). FIG. 2C illustrates an alkaline phosphatase gene.

In some embodiments, the reporter enzyme gene may be a luciferase gene. Examples of luciferase include firefly luciferase and Renilla luciferase. In an embodiment, and as shown in FIG. 1, the reporter enzyme gene may be a modified luciferase gene. The enzyme may have a much stronger signal compared to native or other modified luciferases. The modified luciferase gene may produce NanoLuc® luciferase (nluc; Promega, Madison, Wis., USA). The nluc gene may be inserted into the expression construct as shown in FIG. 1 (see dashed underlining in FIG. 1; nucleotides 278-790 of SEQ ID NO:1). FIG. 2A illustrates the NanoLuc gene.

In some embodiments, the expression constructs include genes for more than one reporter enzyme. In some embodiments, the expression constructs include more than one copy of a given reporter enzyme gene.

The protein affinity tag gene product may enable capture of fusion proteins that include the tag. Captured proteins may be concentrated by providing a relatively small target surface area to which the tag binds. Localizing tags and the fused reporter enzymes may concentrate signals produced by the enzymes, which may increase sensitivity of assays employing tag-fused enzymes compared to assays that do not capture reporter enzymes.

The protein affinity tag gene product may include, but is not limited to, a carbohydrate binding module, a chitin binding protein, glutathione-S-transferase, a His tag, a maltose binding protein, or a Strep-tag. In an embodiment, and as shown in FIG. 1, the protein affinity tag may be a cellulose binding motif. The cellulose binding motif may be CBM2a (cbm) from the xylanase 10A gene of Cellulomonas fimi. The motif may irreversibly bind to crystalline cellulose. The motif may be inserted immediately downstream of the reporter enzyme gene, such as is shown in FIG. 1 (see dot-and-dash underlining in FIG. 1; nucleotides 791-1134 of SEQ ID NO:1; see dot-and-dash underlining in FIG. 1). In the design and construction of the expression construct, a protein affinity tag may help immobilize a resultant fusion protein, which may help increase concentrations and/or improve detection of the protein.

In an embodiment, the reporter enzyme is alkaline phosphatase, or a modified version thereof, and the protein affinity tag is a carbohydrate binding module, and the resultant fusion protein is alkaline phosphatase-carbohydrate binding module. FIG. 2D illustrates an alkaline phosphatase-carbohydrate binding module nucleic acid and FIG. 2E illustrates an alkaline phosphatase protein (second from right) and alkaline phosphatase-carbohydrate binding module fusion protein (right).

In an embodiment, the reporter enzyme is luciferase, or a modified version thereof, and the protein affinity tag is a carbohydrate binding module, and the resultant fusion protein is luciferase-carbohydrate binding module. FIG. 2B illustrates a luciferase-carbohydrate binding module nucleic acid and FIG. 2E illustrates a luciferase protein (left) and luciferase-carbohydrate binding module fusion protein (second from left).

The expression constructs may also include one or more promoters, which may help initiate transcription of the reporter enzyme and protein affinity tag. The promoter may be a phage-specific promoter. In an embodiment, the expression construct is insertable into T7 bacteriophage and the promoter is a T7-specific promoter. For example, and as shown in FIG. 1, the promoter may be phi10 (see single underlining in FIG. 1; nucleotides 159-176 of SEQ ID NO:1).

The expression constructs may also include one or more ribosome binding sites, which may help recruit ribosomes or help initiate protein translation. In an embodiment, and as shown in FIG. 1, a ribosome binding site has the sequence of nucleotides 177-211 of SEQ ID NO:1 (see double underlining in FIG. 1).

The expression constructs may also include one or more leader sequences. The leader sequence may direct the expressed fusion protein to a particular location in the cell, such as the periplasm. In an embodiment, and as shown in FIG. 1, a periplasm-directing leader sequence is the pelB leader sequence (see dot underlining in FIG. 1; nucleotides 212-277 of SEQ ID NO:1).

The expression constructs may also include regions homologous to phage DNA. The homologous regions may include a multiple cloning site of a phage into which the constructs will be inserted. For example, the target phage may be T7 and the multiple cloning site may be positioned immediately downstream of the major capsid gene in gene 10B, between the T7 select left arm and right arm. In an embodiment, and as shown in FIG. 1, expression constructs may include a plurality of nucleotide bases (bases lack underlining in FIG. 1; nucleotides 1-143 and 1135-1217 of SEQ ID NO:1) homologous to a region downstream of the T7 major capsid gene.

The expression constructs may also include at least one stop codon upstream of the reporter gene, which may help permit expression of the reporter enzyme-protein affinity tag fusion protein alone rather than as a fusion with the capsid protein.

The expression constructs may also include non-coding or junk bases. In an embodiment, and as shown in FIG. 1, expression constructs may include about 15 junk bases (bases lack underlining in FIG. 1; nucleotides 144-158 of SEQ ID NO:1).

In some embodiments, the expression construct includes one or more nucleotide bases homologous to the phage multiple cloning site, at least one phage-specific promoter, at least one ribosome binding site, at least one leader sequence, a reporter enzyme gene, and a protein affinity tag gene. In an embodiment, the expression construct includes a plurality of bases homologous to the T7 multiple cloning site, a T7 promoter, a ribosome binding site, a pelB leader sequence, an alkaline phosphatase or modified alkaline phosphate gene, and a carbohydrate binding module gene. In an embodiment, the expression construct includes a plurality of bases homologous to the T7 multiple cloning site, a T7 promoter, a ribosome binding site, a pelB leader sequence, a luciferase or modified luciferase gene, and a carbohydrate binding module gene. In an embodiment, the expression construct has the nucleotide sequence of SEQ ID NO: 1.

### Expression Constructs in Bacteriophages

As used herein, the term “bacteriophage” includes viruses that can infect bacteria (bacteriophages) and viruses that can infect mycobacteria (mycobacteriophages). Bacteriophages generally infect a limited and specific number of strains of a given species of bacteria or mycobacteria (either or both are referred to as “bacteria” herein). Bacteriophages may be employed as bacterial recognition elements at least in part due to their strain-specific infectiveness.

Bacteria infectable by bacteriophages are detectable by the methods and devices disclosed herein. The methods and devices may detect a single species or strain of bacteria, or a plurality of species or strains of bacteria. Examples of bacteria infectable by bacteriophages include, but are not limited to, E. coli and M. tuberculosis.

The expression constructs disclosed herein are insertable into bacteriophages. Expression constructs may be insertable into a single species of bacteriophage or a plurality of species of bacteriophage. Examples of bacteriophages include, but are not limited to, T2, T3, T4, T5, T6, T7, BW-1, HK97, λ, M13, MS2, Qβ, RB69, and Φ174. In an embodiment, an expression construct is insertable into a T7 bacteriophage, which may be as described in Example 3.

### Methods of Capturing Reporter Enzymes for Detection

The expression constructs disclosed herein may be used in methods of immobilizing (capturing) the encoded fusion proteins. Capturing the fusion proteins may help concentrate them, which may thereby concentrate or enhance a detectable signal produced by the fusion proteins.

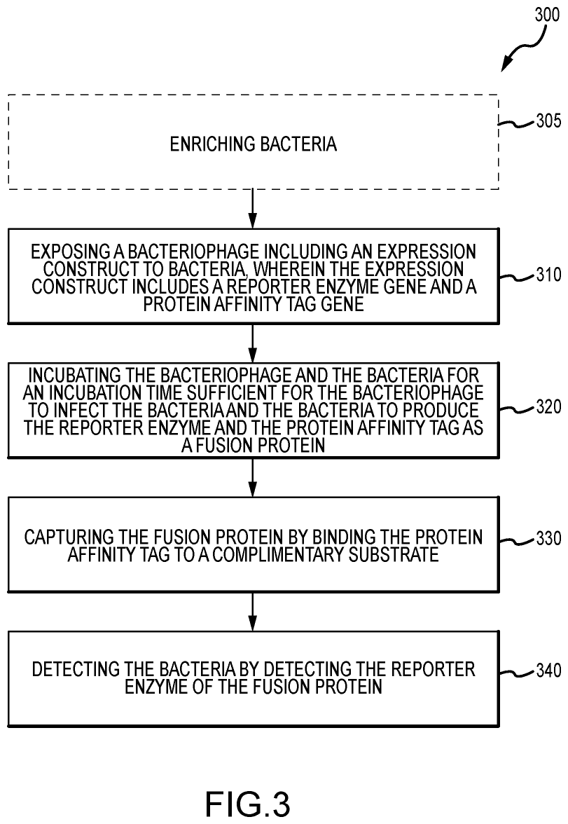

FIG. 3 is a flow chart of a method 300 for capturing bacterial products for detection, according to an embodiment. The method 300 includes an act 310 of exposing a bacteriophage including an expression construct to bacteria, wherein the expression construct includes a reporter enzyme gene and a protein affinity tag gene. The method 300 includes an act 320 of incubating the bacteriophage and the bacteria for an incubation time sufficient for the bacteriophage to infect the bacteria and the bacteria to produce the reporter enzyme and the protein affinity tag as a fusion protein. The method 300 includes an act 330 of capturing the fusion protein by binding the protein affinity tag to a complementary substrate. The method 300 includes an act 340 of detecting the bacteria by detecting the reporter enzyme of the fusion protein. The method 300 may include an optional act 305 of enriching bacteria.

The optional act 305 of enriching bacteria may be included to permit small bacterial colonies to grow large enough to be detectable, which may help minimize false negative results. The act 305 may include incubating bacteria for an enrichment time sufficient for the bacterial colonies to grow large enough to be detectable. The enrichment time may be about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 12 hours, about 4 hours to about 12 hours, about 6 hours to about 12 hours, about 8 hours to about 12 hours, about 2 hours to about 4 hours, about 1 hour to about 3 hours, or about 2 hours.

The optional act 305 may be performed on an enrichment surface on which the bacteria are located. In an embodiment, the enrichment surface is constructed of a material to which a protein affinity tag does not bind. The material may be a non-cellulosic material. The non-cellulosic material may be, for example, polyvinylidene difluoride (PVDF), polycarbonate (PC), tracked-etched polycarbonate (PCTE), polyethersulfone (PES), or tracked-etched polyester (PETE).

The act 310 of exposing a bacteriophage including an expression construct to bacteria, wherein the expression construct includes a reporter enzyme gene and a protein affinity tag gene may include any of the bacteriophages, bacteria, expression constructs, reporter enzymes, and protein affinity tags disclosed herein.

The act 320 of incubating the bacteriophage and the bacteria for an incubation time sufficient for the bacteriophage to infect the bacteria and the bacteria to produce the reporter enzyme and the protein affinity tag as a fusion protein may be performed on an enrichment surface. In an embodiment, the enrichment surface is constructed of a material to which a protein affinity tag does not bind. The material may be a non-cellulosic material. The non-cellulosic material may be, for example, polyvinylidene difluoride (PVDF), polycarbonate (PC), tracked-etched polycarbonate (PCTE), polyethersulfone (PES), or tracked-etched polyester (PETE). In some embodiments that include optional act 305, the enrichment surface may be the same enrichment surface as that on which the bacteria are enriched.

In act 320, the incubation time may be about 10 minutes to about 90 minutes, about 10 minutes to about 80 minutes, about 10 minutes to about 70 minutes, about 10 minutes to about 60 minutes, about 10 minutes to about 50 minutes, about 10 minutes to about 40 minutes, about 10 minutes to about 30 minutes, about 20 minutes to about 90 minutes, about 30 minutes to about 90 minutes, about 40 minutes to about 90 minutes, about 50 minutes to about 90 minutes, about 60 minutes to about 90 minutes, about 30 minutes to about 45 minutes, or less than 60 minutes.

The act 330 of capturing the fusion protein by binding the protein affinity tag to a complementary substrate may help concentrate the fusion protein. Concentrating the fusion proteins may help concentrate or enhance a detectable signal produced by the fusion proteins.

In an embodiment, the complementary substrate is constructed of a material different from the material of the enrichment surface of act 320 and of optional act 305. Constructing the enrichment surface and the complementary substrate of different materials permits concentration of the fusion proteins released from bacteria at a location distinct from the bacteria. In some embodiments, the enrichment surface has a larger area than the complementary substrate, which may help maximize the number of bacteria trapped on the enrichment surface while also maximally concentrating the fusion proteins, and thereby the detectable signals they produce, on the complementary substrate.

In an embodiment, the protein affinity tag includes a cellulose binding motif and the complementary substrate is constructed of a cellulose-based material. Cellulose-based materials include, for example, cellulose acetate, cellulose ester, nitrocellulose, and regenerated cellulose.

The act 340 of detecting the bacteria by detecting the reporter enzyme of the fusion protein may include detecting a product of the reporter enzyme. The product may be visualized with the naked eye, a luminometer, a fluorometer, or a phosphorimeter. A lower detection limit may be about 1 CFU/100 mL to about 100 CFU/100 mL, about 1 CFU/100 mL to about 75 CFU/100 mL, about 1 CFU/100 mL to about 50 CFU/100 mL, about 1 CFU/100 mL to about 25 CFU/100 mL, or about 1 CFU/100 mL to about 10 CFU/100 mL.

### Methods of Detecting Bacteria Using Expression Constructs

The expression constructs disclosed herein may be used in methods of detecting bacteria in a sample. The methods may provide fast, sensitive, and accurate detection of one or more species or strains of bacteria.

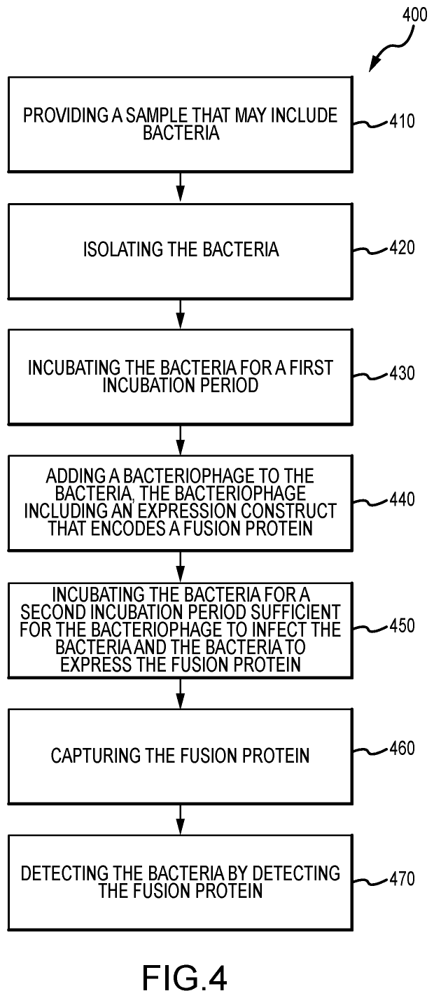

FIG. 4 is a flow chart of a method 400 for detecting bacteria, according to an embodiment. The method 400 includes an act 410 of providing a sample that may include bacteria. The method 400 includes an act 420 of isolating the bacteria. The method 400 includes an act 430 of incubating (enriching) the bacteria for a first incubation period. The method 400 includes an act 440 of adding a bacteriophage to the bacteria, the bacteriophage including an expression construct that encodes a fusion protein. The method 400 includes an act 450 of incubating the bacteria for a second incubation period sufficient for the bacteriophage to infect the bacteria and the bacteria to express the fusion protein. The method 400 includes an act 460 of capturing the fusion protein. The method 400 includes an act 470 of detecting the bacteria by detecting the fusion protein.

In the act 410 of providing a sample that may include bacteria, the sample may be a fluid such as drinking water, postharvest rinse water, environmental water, beverages, or urine. The bacteria may be any bacteria described above.

In the act 420 of isolating the bacteria, the bacteria may be isolated on an enrichment surface. The enrichment surface may be constructed of a non-cellulosic material, as described above.

The act 430 of incubating the bacteria for a first incubation period may include permitting a metabolic activity of the bacteria to increase and/or permitting a number of cells of bacteria to increase. The first incubation period may be about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 12 hours, about 4 hours to about 12 hours, about 6 hours to about 12 hours, about 8 hours to about 12 hours, about 2 hours to about 4 hours, or about 1 hour to about 3 hours.

The act 440 of adding a bacteriophage to the bacteria, the bacteriophage including an expression construct that encodes a fusion protein may include any bacteriophage, expression construct, and fusion protein described above.

In the act 450 of incubating the bacteria for a second incubation period sufficient for the bacteriophage to infect the bacteria and the bacteria to express the fusion protein, the second incubation period may be about 10 minutes to about 90 minutes, about 10 minutes to about 80 minutes, about 10 minutes to about 70 minutes, about 10 minutes to about 60 minutes, about 10 minutes to about 50 minutes, about 10 minutes to about 40 minutes, about 10 minutes to about 30 minutes, about 20 minutes to about 90 minutes, about 30 minutes to about 90 minutes, about 40 minutes to about 90 minutes, about 50 minutes to about 90 minutes, about 60 minutes to about 90 minutes, about 30 minutes to about 45 minutes, or less than 60 minutes.

The act 460 of capturing the fusion protein may help concentrate the fusion protein. Concentrating the fusion proteins may help concentrate or enhance a detectable signal produced by the fusion proteins.

In an embodiment, capturing the fusion protein may include capturing by an interaction between a protein affinity tag of the fusion protein and a complementary substrate. The complementary substrate may be constructed as described above for act 330.

The act 470 of detecting the bacteria by detecting the fusion protein may include detecting a product produced by the fusion protein. The product may be visualized with the naked eye, a luminometer, a fluorometer, or a phosphorimeter. A lower detection limit may be about 1 CFU/100 mL to about 100 CFU/100 mL, about 1 CFU/100 mL to about 75 CFU/100 mL, about 1 CFU/100 mL to about 50 CFU/100 mL, about 1 CFU/100 mL to about 25 CFU/100 mL, or about 1 CFU/100 mL to about 10 CFU/100 mL.

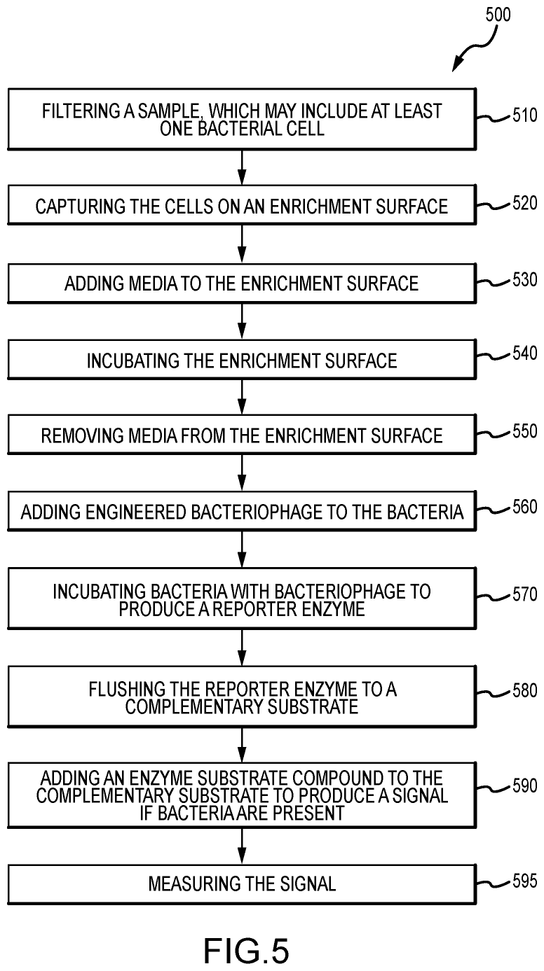

FIG. 5 is a flow chart of a method 500 for detecting bacteria, according to an embodiment. The method 500 includes an act 510 of filtering a sample, which may include at least one bacterial cell. The method 500 includes an act 520 of capturing the cells on an enrichment surface. The method 500 includes an act 530 of adding media to the enrichment surface. The method 500 includes an act 540 of incubating the enrichment surface. The method 500 includes an act 550 of removing media from the enrichment surface. The method 500 includes an act 560 of adding engineered bacteriophage to the bacteria. The method 500 includes an act 570 of incubating bacteria with the bacteriophage to produce a reporter enzyme. The method 500 includes an act 580 of flushing the reporter enzyme to a complementary substrate. The method 500 includes an act 590 of adding an enzyme substrate compound to the complementary substrate to produce a signal if bacteria are present. The method 500 includes an act 595 of measuring the signal.

In the act 510 of filtering a sample, which may include at least one bacterial cell, the sample may be a fluid such as drinking water, postharvest rinse water, environmental water, beverages, or urine. The bacteria may be any bacteria described above. The volume of the sample may be about 100 mL.

In the act 520 of capturing the cells on an enrichment surface, the enrichment surface may be constructed of a non-cellulosic material, as described above.

In the act 530 of adding media to the enrichment surface, the media may be Luria Bertani (LB) broth or any other media in which bacteria may be cultured.

The act 540 of incubating the enrichment surface may permit bacteria to grow and multiply, such as to detectable levels. The incubation temperature may be about 37° C. The enrichment surface may be incubated for about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 12 hours, about 4 hours to about 12 hours, about 6 hours to about 12 hours, about 8 hours to about 12 hours, about 2 hours to about 4 hours, about 1 hour to about 3 hours, or about 2 hours.

The act 550 of removing media from the enrichment surface may include applying a vacuum source, directly or indirectly, to the enrichment surface.

The act 560 of adding engineered bacteriophage to the bacteria may include any bacteriophage or bacteria described above.

In the act 570 of incubating bacteria with the bacteriophage to produce a reporter enzyme, the incubation temperature may be about 37° C. The bacteria may be incubated for about 10 minutes to about 90 minutes, about 10 minutes to about 80 minutes, about 10 minutes to about 70 minutes, about 10 minutes to about 60 minutes, about 10 minutes to about 50 minutes, about 10 minutes to about 40 minutes, about 10 minutes to about 30 minutes, about 20 minutes to about 90 minutes, about 30 minutes to about 90 minutes, about 40 minutes to about 90 minutes, about 50 minutes to about 90 minutes, about 60 minutes to about 90 minutes, about 30 minutes to about 45 minutes, or less than 60 minutes.

The act 580 of flushing the reporter enzyme to a complementary substrate may include flushing with a fluid such as LB or phosphate buffered saline. The complementary substrate may be constructed as described above for act 330.

The act 590 of adding an enzyme substrate compound to the complementary substrate to produce a signal if bacteria are present may include any substrate compound that may be acted upon by the reporter enzyme to produce a detectable signal. In an embodiment, the reporter enzyme is luciferase and the enzyme substrate compound is luciferin. Oxidation of luciferin by luciferase produces bioluminescence. In an embodiment, the reporter enzyme is alkaline phosphatase and the enzyme substrate compound is 5-bromo-4-chloro-3-indolyl-phosphatase, p-toluidine salt (BCIP). Alkaline phosphatase hydrolysis of BCIP leads to a colored precipitate.

The act 595 of measuring the signals may include visualizing the signals with, for example, the naked eye, a luminometer, a fluorometer, or a phosphorimeter. The signals may be quantified manually or with the aid of a software program.

### Devices for Performing Methods

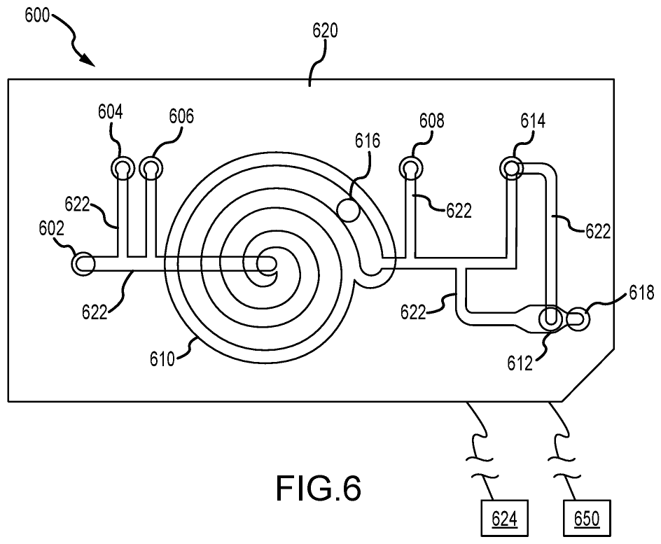

Any of the methods 300, 400, 500 described above may be performed by or on a device, which may be a microfluidic device. FIG. 6 is a schematic of a microfluidic device 600, according to an embodiment. The device 600 may be generally understood as including a plurality of immobilization regions positioned between a plurality of inlets and outlets. Fluids, such as samples to be assayed or reagents, may enter through the inlets. The fluids may exit through the outlets, as may gases. Some or all of the fluids may pass through or may be retained in one or more of the immobilization regions.

The immobilization regions may include a first immobilization region 610 and a second immobilization region 612. The inlets may include a first inlet 602, a second inlet 604, a third inlet 606, and a fourth inlet 608. The outlets may include a first outlet 614, a second outlet 616, and a third outlet 618. Some or all of the immobilization regions 610, 612; inlets 602, 604, 606, 608; and outlets 614, 616, 618 may be mounted on a backing 620.

The backing 620 may help provide structural support for these or other components and/or may help maintain these or other components in a single physical location.

The first inlet 602 may be fluidly connected, such as by a channel 622, to the first immobilization region 610. The first inlet 602 may be positioned upstream of the first immobilization region 610. In some embodiments, the first inlet 602 is configured to accept a fluid sample to be assayed. The sample may be, for example, drinking water or postharvest rinse water. The sample may include bacteria.

The second inlet 604 may be fluidly connected, such as by a channel 622, to the first immobilization region 610. The second inlet 604 may be positioned upstream of the first immobilization region 610. In some embodiments, the second inlet 604 is configured to accept media. The media may be Luria Bertani (LB) broth or any other media in which bacterial may be cultured.

The third inlet 606 may be fluidly connected, such as by a channel 622, to the first immobilization region 610. The third inlet 606 may be positioned upstream of the first immobilization region 610. In some embodiments, the third inlet 606 is configured to accept bacteriophage. The bacteriophage may be any bacteriophage described above.

The fourth inlet 608 may be fluidly connected, such as by a channel 622, to the second immobilization region 612. The fourth inlet 608 may be positioned upstream of the second immobilization region 612 and/or downstream of the first immobilization region 610. In some embodiments, the fourth inlet 608 is configured to accept a substrate compound for a reporter enzyme. The substrate compound may be any substrate compound described above.

The first immobilization region 610 may be a region on which bacteria are permitted to grow and multiply. In an embodiment, the first immobilization region 610 is constructed of a material to which bacteria may adhere but to which a protein affinity tag does not bind. The material may be a non-cellulosic material. The non-cellulosic material may be, for example, polyvinylidene difluoride (PVDF), polycarbonate (PC), tracked-etched polycarbonate (PCTE), polyethersulfone (PES), or tracked-etched polyester (PETE).

The second immobilization region 612 may be a region capable of binding a protein affinity tag. In an embodiment, the second immobilization region 612 is constructed of a material different from the material of the first immobilization region 610. Constructing the first immobilization region 610 and the second immobilization region 612 of different materials permits concentration of the fusion proteins released from bacteria at a location distinct from the bacteria. In some embodiments, the first immobilization region 610 has a larger area than the second immobilization region 612, which may help maximize the number of bacteria trapped on the first immobilization region 610 while also maximally concentrating the fusion proteins, and thereby the detectable signals they produce, on the second immobilization region 612.

In an embodiment, the second immobilization region 612 is constructed of a cellulose-based material and the protein affinity tag includes a cellulose binding motif. Cellulose-based materials include, for example, cellulose acetate, cellulose ester, nitrocellulose, and regenerated cellulose.

The first outlet 614 may be fluidly connected, such as by a channel 622, to the first immobilization region 610. The first outlet 614 may be positioned downstream of the first immobilization region 610. In some embodiments, the first outlet 614 is connectable to a vacuum source. Application of a vacuum source to the first outlet 614 may help draw one or more fluids through the device 600. The fluid may be, for example, LB media.

Each of the second outlet 616 and third outlet 618 may be vent outlets, which may help fluids, including gases, move through and/or exit the device 600. The second outlet 616 may be fluidly connected, such as by a channel 622, to the first immobilization region 610. The second outlet 616 may be positioned in the first immobilization region 610. The third outlet 618 may be fluidly connected, such as by a channel 622, to the second immobilization region 612. The second outlet 616 may be positioned downstream of the second immobilization region 612.

The device 600 may include one or more valves (not shown) for controlling fluid flow from an inlet and/or to an outlet. Examples of devices that include valves are described in U.S. patent application Ser. No. 15/870,370, which is hereby incorporated herein by reference in its entirety to the extent not inconsistent herein.

In an embodiment, the device 600 includes or is operably associated with an electronic controller 650 that can control the operation of one or more components and/or functions of the device 600. For example, the electronic controller 650 that includes electronic circuity can be coupled to any one or more of the first inlet 602, second inlet 604, third inlet 606, and fourth inlet 608 and control entry of fluid into any of the inlets 602, 604, 606, 608. The electronic controller 650 may be coupled to any one or more of the first outlet 614, second outlet 616, and third outlet 618 and control exit of fluid from any of the outlets 614, 616, 618. The electronic controller 650 may be coupled to the detector 624 and control detection of a signal, such as one produced in the second immobilization region 612.

Generally, the controller 650 can include control electrical circuitry that forms at least part of a processor, memory, storage, and input/output (I/O) interface. The controller 650 can be configured or programed to perform one or more acts or steps as described herein. It should be also appreciated that the controller 650 can be or can include a general purpose computer that can be programmed or can include instructions to perform the acts described herein. Additionally or alternatively, the controller 650 can be configured as a special purpose controller 650 (e.g., the controller 650 can include programmable field gate arrays (PFGA) that can be programmed or configured, such that the controller 650 can perform the acts described herein).

In an example of the use and operation of the device 600, a fluid sample, which may include bacteria, is introduced to the first inlet 602. The sample may be introduced by, for example, pipette or syringe. The sample may flow through a channel 622 to the first immobilization region 610. The channel 622 may or may not include a valve.

Bacteria in the sample may adhere to the first immobilization region 610. Fluid in the sample may travel, passively or with the assistance of an applied vacuum force, through one or more channels 622 to an outlet, such as the first outlet 614.

Cell culture media may be introduced to the second inlet 604. The fluid may be introduced by, for example, pipette or syringe. The sample may flow through a channel 622 to the first immobilization region 610. The channel 622 may or may not include a valve.

The first immobilization region 610 or the device 600 may be incubated under conditions that promote bacterial growth. For example, the first immobilization region 610 or the device 600 may be incubated at 37° C. for any enrichment time described above.

Bacteriophage in a suitable carrier fluid may be introduced to the third inlet 606. The carrier fluid may be introduced by, for example, pipette or syringe. The sample may flow through a channel 622 to the first immobilization region 610. The channel 622 may or may not include a valve.

The bacteriophage may be permitted to infect bacteria that may be present in the first immobilization region 610. The bacteriophage may be incubated with the bacteria for any amount of time as described above. Infection may lead to bacterial cell lysis and thereby release of reporter enzyme-affinity binding tag fusion proteins. The proteins may travel, passively or with the assistance of an applied vacuum force, through one or more channels 622 to the second immobilization region 612. The proteins may travel passively or may be flushed with a fluid, such as cell culture media or phosphate buffered saline. The proteins may be captured on the second immobilization region 612, such as by interaction between an affinity binding tag and a complementary material of the second immobilization region 612.

A substrate compound for a reporter enzyme in a suitable carrier fluid may be introduced to the second inlet 604. The carrier fluid may be introduced by, for example, pipette or syringe. The substrate compound may flow through a channel 622 to the second immobilization region 612. The channel 622 may or may not include a valve.

The second immobilization region 612 or the device 600 may be incubated under conditions that promote development of a detectable signal, as described above.

The detectable signal may be detected by a detector 624, which may be incorporated into the device 600 or may be external to the device 600. The detector 624 may be the naked eye, a magnifying lens, a luminometer, a fluorometer, or a phosphorimeter.

### Kits

Any two or more of a media for culturing bacteria, bacteriophage into which an expression construct has been inserted, a substrate compound for a reporter enzyme, and a device for detecting bacteria may be combined to form a kit.

The media may be any media described above.

The bacteriophage and expression construct may be any bacteriophage and expression construct, respectively, described above. In some embodiments, the bacteriophage may be provided in lyophilized form. Lyophilization may increase the shelf-life of the bacteriophage compared to bacteriophage stored in a fluid. In some embodiments, sterile water is included in the kits. Sterile water may be used to reconstitute lyophilized bacteriophage. In some embodiments, more than one type of bacteriophage is included and the different types of bacteriophage are capable of infecting different species or strains of bacteria. The plurality of types of bacteriophage may be mixed together or maintained separately. Including a plurality of types of bacteriophage helps enable detection of more than one species or strain of bacteria. The substrate compound for a reporter enzyme may be any substrate compound described above.

The following working examples provide further detail in connection with the specific embodiments described above.

### EXAMPLES

### Example 1—Reporter Enzyme Expression Constructs

Double stranded DNA cassette fragments were synthesized by IDT (Coralville, Iowa, USA). One expression cassette included a bacterial alkaline phosphatase gene (alp) mutated to produce two amino acid substitutions (D153G/D330N), which results in a modified phosphatase enzyme with activity increased by more than two orders of magnitude (Muller et al, 2001, Chembiochem 2(7-8), 517-523). A second expression cassette, shown in FIG. 1, included a modified luciferase gene (NanoLuc®; Promega, Madison, Wis., USA; nluc; nucleotides 278-790 of SEQ ID NO:1; see dashed underlining in FIG. 1) capable of generating a luciferase enzyme with a much stronger signal compared to other commonly employed luciferases (Hall et al, 2012, ACS Chem Bio 7(11), 1848-1857).

An affinity binding module (nucleotides 791-1134 of SEQ ID NO:1; see dot-and-dash underlining in FIG. 1) with irreversible binding to crystalline cellulose (CBM2a) from the xylanase 10A gene from Cellulomonas fimi (cbm; McLean et al, 2000, Prot Eng 13(11), 801-809) was inserted 3′ to each reporter enzyme gene. The binding module enables immobilization of the resulting reporter enzyme fused to the N-terminus of the module.

Each of a strong T7 promoter (phi10; nucleotides 159-176 of SEQ ID NO:1; see single underlining in FIG. 1) and custom ribosome binding site (nucleotides 177-211 of SEQ ID NO:1; see double underlining in FIG. 1; Tian and Salis, 2015, Nucleic Acids Res 43(14), 7137-7151) was inserted upstream of the enzymes to help promote high levels of expression. A pelB leader sequence (nucleotides 212-277 of SEQ ID NO:1; see dot underlining in FIG. 1) was inserted between the ribosome binding site and the enzymes to direct the expressed enzyme to the cell periplasm.

Each expression cassette was flanked by regions homologous to the phage multiple cloning site, which is immediately downstream of the major capsid gene in gene 10B, between the T7 select left arm and right arm. Each cassette included a stop codon upstream of the reporter, which permitted expression of the reporter-cellulose binding fusion protein alone rather than as a fusion with the capsid protein. A few junk bases followed the gene 10B sequence and preceded the T7 promoter. The homologous and junk bases are not underlined in FIG. 1.

### Example 2—Preparation of Bacteria, Phages, and Phage DNA

E. coli BL21 was obtained from ATCC (Manassas, Va. USA) and E. coli ECOR #13, a strain isolated from a healthy human, was obtained from the Thomas S. Whittam STEC Center (East Lansing, Mich., USA). Bacterial cultures were initially stored at −80° C. in 25% glycerol prior to use. Cultures were grown in Luria Bertani (LB) broth and plated on LB agar. Overnight cultures of E. coli were prepared in 10 mL of LB inoculated with a single bacterial colony and incubated (37° C., 200 rpm, 18 hr). Serial dilutions were performed in sterile phosphate buffer saline (PBS).

Exponentially growing E. coli host cells (200 mL) were infected with the lytic coliphage T7 Select 415-1 (Millipore Sigma, Burlington, Mass., USA) at an MOI of 0.1 until cellular lysis caused a significant decrease in OD₆₀₀ (1.5-2 h). Low speed centrifugation was used to clear cellular debris (3,200×g, 10 min, 4° C.) before sterile filtration (0.22 μm). Polyethylene glycol 6000 (PEG6000; 4%) and sodium chloride (NaCl; 0.4M) were added and incubated overnight at 4° C. to precipitate phage particles. Phage were pelleted by ultracentrifugation (35,000×g, 120 min, 4° C.), resuspended in phosphate buffered saline (PBS; pH 7.4), enumerated by standard double overlay plaque assays, and stored at 4° C. All phage used in detection assays were diluted to 1×10⁹ PFU/mL in LB, sterile filtered (0.22 μm), and stored at 4° C. as phage stock solutions.

Lysates of T7 Select 415-1 bacteriophage (>10¹¹ PFU/mL) were used for genome extraction and purification. The phage stock solution was treated with sodium dodecyl sulfate (SDS; 2%) for 20 min. at 70° C. to disrupt the capsid and release phage genomic DNA. After cooling on ice, DNA was precipitated with sodium acetate (0.3 M) and ethanol (70%). The sample was centrifuged (10 min, 10,000×g, 4° C.) and the supernatant was passed through a Genomic Tip 100/G (Qiagen) according to the manufacturer's recommendations.

### Example 3—Production and Isolation of Reporter Phages

Purified phage DNA was digested with HindIII to prepare the vector for reporter gene insertion. The reporter gene containing homology to each vector arm as described in Example 1, was added to the phage genomic vector at a 2:1 molar ratio and was assembled using NEBuilder® Hifi DNA Assembly Master Mix (NEB, Ipswitch, Mass.). Transformations were performed in electrocompetent E. coli DH10B (MegaX, ThermoFisher) in 1-mm cuvettes under standard conditions. Recovery was performed in SOB with shaking until visible signs of lysis occurred. Serial dilutions were performed until double overlay plaque assays revealed individual plaques. Correct clones were identified with application of enzymatic substrate compounds and imaging. Positive plaques were further evaluated using PCR to verify insert size and full genome sequencing. Sanger sequencing results evidenced the correct size insertions, without any mutations, insertions, or deletions within the insertion site. Full genome sequencing revealed no significant mutations in the remainder of the genome. No observable differences in plaque morphology, burst size, and/or lysis times were detected between the wild-type and recombinant phages.

Schematics of the native and engineered nucleic acids and proteins and associated phages are shown in FIG. 2. FIG. 2A illustrates the NanoLuc nucleic acid and associated reporter phage, T7_{NL}; FIG. 2B illustrates the NanoLuc fusion with a carbohydrate binding module and the associated reporter phage, T7_{NLC}; FIG. 2C illustrates the alkaline phosphatase nucleic acid and associated reporter phage, T7_{AL}; and FIG. 2D illustrates the alkaline phosphate fusion with a carbohydrate binding module and the associated reporter phage, T7_{ALC}. FIGS. 2A-2D are drawn to scale. FIG. 2E shows scale representations of the resulting NanoLuc protein (Protein Database File 5ibo; left) from FIG. 2A, the NanoLuc-CBM2a fusion protein (CBM2a Protein Database File 1exg; second from left) from FIG. 2B, the alkaline phosphatase protein (Protein Database File 1kh7; second from right) from FIG. 2C, and the alkaline phosphatase-CBM2a fusion protein (right) from FIG. 2D.

### Example 4—Detection of Bacteria by Reporter Enzyme Expression

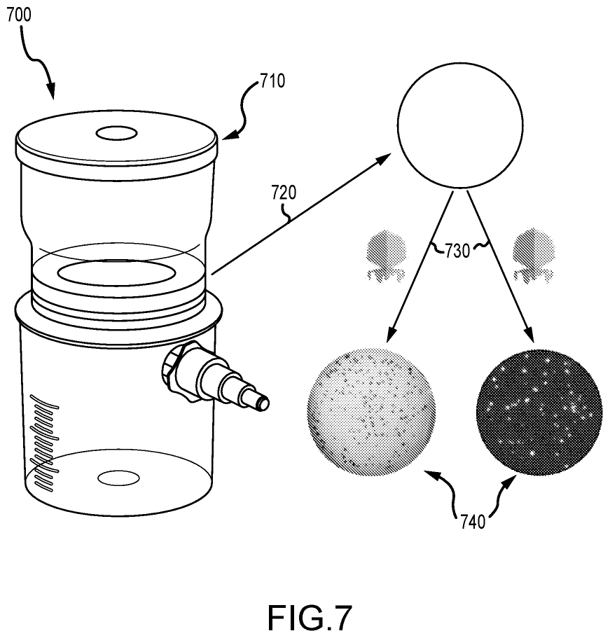

FIG. 7 is a schematic of a bacterial detection method 700 including an act 710 of filtering contaminated fluid samples to capture bacteria on a filter, an act 720 of incubating the captured bacteria, an act 730 of adding phage to permit phage infection of bacteria on the filter, and then an act 740 of imaging of the filter. Drinking water samples (100 mL) were obtained from a municipal water source (Ithaca, N.Y., USA) and autoclaved. The sterile drinking water samples were inoculated with varying concentrations of ECOR #13 and vacuum filtered through a cellulose filter membrane (47 mm diameter, 0.22 μm pore size, Sartorius Stedim Biotech GmbH, Goettingen, Germany) housed in a disposable funnel (Nalgene™, Waltham, Mass.). Following filtration, the filter membrane was removed and placed onto an absorbent pad saturated with LB broth. The filters were incubated (37° C., 8-12 hours) to allow for colony growth. Following the initial enrichment, a phage solution (2 mL, 10⁹ PFU/mL in LB) was applied to the filter and incubated (37° C., 90 min) to initiate phage infection and reporter probe expression.

After brief drying on a sterile absorbent pad, either the phosphatase substrate compound 5-bromo-4-chloro-3-indolyl-phosphatase, p-toluidine salt (BCIP) or the luciferase substrate compound, NanoGlo buffer, (˜300 μL) was applied directly to the filter. BCIP (20 mg/mL N,N-dimethylformamide (DMF)) was stored at −20° C. and diluted tenfold (2 mg/mL) in diethanolamine buffer (1 M DEA, pH 10.1) immediately before use. NanoGlo buffer (Promega, Madison, Wis., USA) was prepared according to the manufacturer's recommendations immediately before use.

Substrate compounds were incubated briefly (37° C., 10 min) to permit colorimetric (BCIP) or bioluminescent (NanoGlo) signal development for imaging.

The reaction product between alkaline phosphatase and BCIP is an insoluble blue precipitate that is easily visualized by the naked eye. Colorimetric images were captured with a DSLR camera on an LED light box (AGPTek, Brooklyn, N.Y., USA), which helped provide the greatest contrast between colonies.

The NanoLuc enzyme, when complexed with its substrate compound NanoGlo, exhibits a blue luminescent signal with a peak emission at 460 nm. Bioluminescent images were captured with a DSLR camera (Rebel T6, Canon, Melville N.Y., USA) in a dark box (LTE-13, Newport Corporation, Irvine, Calif., USA), which helped decrease background light interference, using 30-second exposure times. To mitigate signal decay, the camera was placed as close as possible to the filters. Bioluminescent images were then analyzed using ImageJ (National Institutes of Health, Bethesda, Md., USA). Spot sizes and distribution were determined using ImageJ particle size distribution. Background was relatively low, which helped permit pixel intensities to be multiplied by three and thereby improved visualization of the spots. The spots were also counted visually to determine accuracy of the image analysis.

Results are shown in FIG. 7. Both colorimetric signals (act 740, left side) and bioluminescent signals (act 740, right side), produced from T7_{ALC} and T7_{NLC}, respectively, recombinant phage infection of E. coli captured and cultured on filters were clearly visible.

### Example 5—Determination of Enrichment Time

Bacteria on a filter is detectable after a colony has grown large enough to produce a detectable signal. The growth (enrichment) time depends on numerous factors including generation time, growth conditions, and reporter enzyme kinetics. Enrichment time was studied for the phage-based methods of Example 4.

ECOR #13 colony formation was visible in less than 8 hours (data not shown). Eight hours was selected as the target enrichment time for Example 7, described below, to help ensure that smaller colonies grew large enough to produce a signal, thereby minimizing false negative results. In subsequent assays, the enrichment time was reduced to about 2 hours.

### Example 6—Immobilization of Reporter Enzymes

To evaluate the binding affinity of the CBM fusion proteins for cellulose, phage lysates (1 mL) including each of the reporter enzymes described in Example 1 were slowly spotted onto the center of a cellulose filter and allowed to passively diffuse to completely saturate the filter. After brief drying, the appropriate substrate compound was applied and images were taken as described above for Example 4.

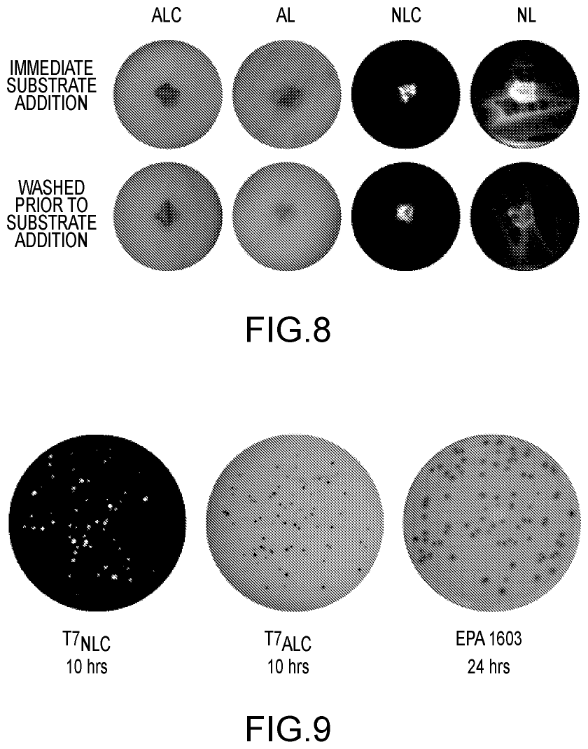

Results are shown in FIG. 8. T7_{AL} (second column from left) and T7_{NL} (right column) phage lysates each exhibited extensive diffusion away from the filter center and signal dilution. T7_{ALC} (left column) and T7_{NLC} (second column from right) phage lysates each displayed limited diffusion with a bright, concentrated signal. Results were similar when the filters were washed with PBS prior to substrate compound addition (bottom row). The results demonstrate that each reporter enzyme, alkaline phosphatase or luciferase, when fused to the carbohydrate binding module, exhibited significantly limited diffusion across the filter compared to the reporter enzyme alone. Binding affinity of the CBM fusion proteins to the cellulose filter is sufficient to limit diffusion across the filter.

### Example 7—Visual Comparison of Phage-Based Detection Methods with EPA Method

The current standard for the detection and enumeration of E. coli bacteria in ambient waters and disinfected wastewaters is EPA Method 1603. The presently disclosed phage-based (T7_{NLC} and T7_{ALC}) methods of bacterial detection and enumeration were compared in parallel to the EPA method.

Drinking water samples (100 mL) were spiked with varying concentrations of E. coli (ECOR #13). Samples were passed through a filter membrane by vacuum filtration as described in Example 4. Each dilution was run in triplicate.

For EPA Method 1603, filters were incubated on modified mTEC (membrane-thermotolerant E. coli) agar for 24 hours. For phage-based methods, filters were incubated for 8 hours (based on the results of Example 5), followed by a 90-minute phage infection period and then 15 minutes of substrate compound addition and imaging. From initial filtration to final results, the total assay time for the phage-based methods was approximately 10 hours compared to approximately 24 hours for the EPA method.

Images of representative filters are shown in FIG. 9. The images demonstrate that the phage-based methods produce comparable results to EPA Method 1603 in less than half the time (10 hours vs. 24 hours).

### Example 8—Statistical Comparison of Phage-Based Detection Methods with EPA Method

The colony forming unit (CFU) counts from each E. coli dilution prepared in Example 7, as well as a negative control containing no inoculated E. coli, were compared to determine agreement between the three tested methods.

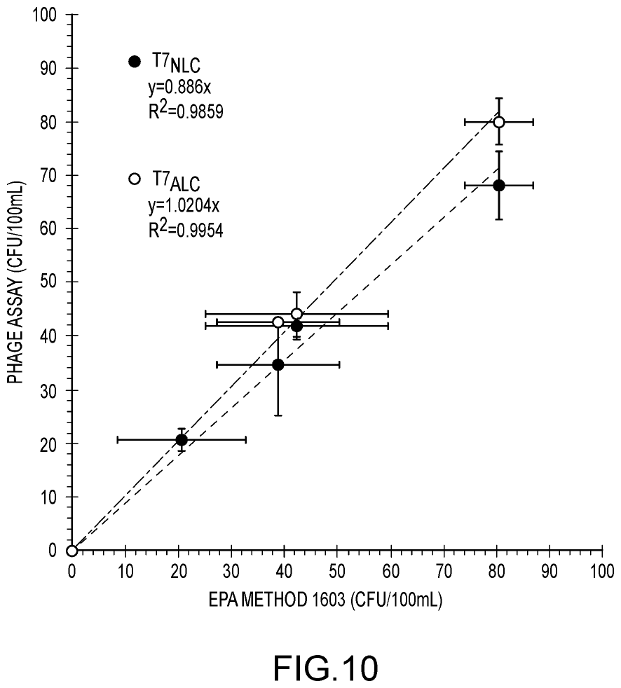

Results are plotted in FIG. 10. Error bars represent the standard deviation of three replicates. The comparison of the T7_{NLC} phage-based method to EPA Method 1603 yielded a linear relationship with a slope of 0.89 (R²=0.99). There was no significant difference at the p<0.01 level between the T7_{NLC} method and EPA Method 1603 [two-factor ANOVA, F(1,3)=1.105, p=0.306]. The comparison of the T7_{ALC} phage-based method to EPA Method 1603 yielded a linear relationship with a slope of 1.02 (R²=1.00). There was no significant difference at the p<0.01 level between the T7_{ALC} method and EPA Method 1603 [two-factor ANOVA, F(1,3)=1.667, p=0.689]. Variations observed within the phage-based methods fell well within the variation of the EPA method. The phage-based methods produce comparable results to EPA Method 1603 in less than half the time.

The state of the art has progressed to the point where there is little distinction left between hardware, software (e.g., a high-level computer program serving as a hardware specification), and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software (e.g., a high-level computer program serving as a hardware specification), and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software (e.g., a high-level computer program serving as a hardware specification) implementation; or, yet again alternatively, the implementer may opt for some combination of hardware, software (e.g., a high-level computer program serving as a hardware specification), and/or firmware in one or more machines, compositions of matter, and articles of manufacture, limited to patentable subject matter under 35 U.S.C. § 101. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary.

In some implementations described herein, logic and similar implementations may include computer programs or other control structures. Electronic circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software (e.g., a high-level computer program serving as a hardware specification) or firmware, or of gate arrays or programmable hardware, such as by performing a reception of or a transmission of one or more instructions in relation to one or more operations described herein. Alternatively or additionally, in some variants, an implementation may include special-purpose hardware, software (e.g., a high-level computer program serving as a hardware specification), firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or invoking circuitry for enabling, triggering, coordinating, requesting, or otherwise causing one or more occurrences of virtually any functional operation described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit).

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood that each function and/or operation within such block diagrams, flowcharts, or examples can be implemented individually and/or collectively, by a wide range of hardware, software (e.g., a high-level computer program serving as a hardware specification), firmware, or virtually any combination thereof, limited to patentable subject matter under 35 U.S.C. 101. In an embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, limited to patentable subject matter under 35 U.S.C. 101, and that designing the circuitry and/or writing the code for the software (e.g., a high-level computer program serving as a hardware specification) and or firmware would be well within the skill of one of skill in the art in light of this disclosure. The mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, the various aspects described herein which can be implemented, individually and/or collectively, by a wide range of hardware, software (e.g., a high-level computer program serving as a hardware specification), firmware, and/or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.), and/or electrical circuitry forming a communications device (e.g., a modem, communications switch, optical-electrical equipment, etc.). The subject matter described herein may be implemented in an analog or digital fashion or some combination thereof.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

The herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A device for concentrating bacteria and bacterial products, the device comprising:
a first inlet fluidly coupled to a first immobilization region, the first immobilization region constructed of a material configured to concentrate the bacteria;
a second inlet fluidly coupled to the first immobilization region;
a third inlet fluidly coupled to the first immobilization region;
a fourth inlet fluidly coupled to a second immobilization region, the second immobilization region constructed of a material configured to concentrate bacterial products and different from the material of the first immobilization region.

2. The device of claim 1, wherein the material of the first immobilization region includes a non-cellulosic material.

3. The device of claim 2, wherein the non-cellulosic material includes polyvinylidene difluoride.

4. The device of claim 1, wherein a surface area of the first immobilization region is greater than a surface area of the second immobilization region.

5. The device of claim 1, wherein the bacterial products include a protein affinity tag.

6. The device of claim 5, wherein the material of the first immobilization region is unable to bind the protein affinity tag.

7. The device of claim 5, wherein the protein affinity tag includes a cellulose binding motif.

8. The device of claim 7, wherein the material of the second immobilization region includes a cellulose-based material.

9. The device of claim 1, wherein the first inlet is configured to accept a sample including bacteria.

10. The device of claim 1, wherein the second inlet is configured to accept media for culturing the bacteria.

11. The device of claim 1, wherein the third inlet is configured to accept bacteriophage capable of infecting the bacteria.

12. The device of claim 1, wherein the bacterial products include a reporter enzyme and the fourth inlet is configured to accept a substrate compound for the reporter enzyme.

13. The device of claim 1, wherein at least one of the first inlet fluidly coupled to the first immobilization region, the second inlet fluidly coupled to the first immobilization region, the third inlet fluidly coupled to the first immobilization region, and the fourth inlet fluidly coupled to the second immobilization region is fluidly coupled without a valve.

14. The device of claim 1, wherein at least one of the first inlet fluidly coupled to the first immobilization region, the second inlet fluidly coupled to the first immobilization region, the third inlet fluidly coupled to the first immobilization region, and the fourth inlet fluidly coupled to the second immobilization region is fluidly coupled with a valve.

15. The device of claim 1, further comprising at least one of a first outlet fluidly coupled to the first immobilization region, a second outlet fluidly coupled to the first immobilization region, and a third outlet fluidly coupled to the second immobilization region.

16. The device of claim 15, wherein at least one of the first outlet fluidly coupled to the first immobilization region, the second outlet fluidly coupled to the first immobilization region, and the third outlet fluidly coupled to the second immobilization region is fluidly coupled without a valve.

17. The device of claim 15, wherein at least one of the first outlet fluidly coupled to the first immobilization region, the second outlet fluidly coupled to the first immobilization region, and the third outlet fluidly coupled to the second immobilization region is fluidly coupled with a valve.

18. A method of detecting bacteria, the method comprising:
providing a sample including bacteria;
isolating the bacteria;
incubating the bacteria for a first incubation period;
adding a bacteriophage to the bacteria, the bacteriophage including an expression construct that encodes a fusion protein;
incubating the bacteria for a second incubation period sufficient for the bacteriophage to infect the bacteria and the bacteria to express the fusion protein;
capturing the fusion protein; and
detecting the bacteria by detecting the fusion protein.

19. The method of claim 18, wherein the method is performed on a device, the device including:
a first inlet for receiving the sample including bacteria;
a first immobilization region for isolating the bacteria, incubating the bacteria for the first incubation period, and incubating the bacteria for the second incubation period sufficient for the bacteriophage to infect the bacteria and the bacteria to express the fusion protein;
a third inlet for receiving bacteriophage to be added to the bacteria; and
a second immobilization region for capturing the fusion protein.

20. The method of claim 18, wherein the fusion protein includes a reporter enzyme linked to a protein affinity tag.

21. The method of claim 20, wherein capturing the fusion protein includes capturing the fusion protein by an interaction between the protein affinity tag and a complementary substrate.

22. The method of claim 18, wherein during the first incubation period, at least one of a metabolic activity or a number of cells of the bacteria increases.

23. The method of claim 18, wherein the first incubation period is for less than about 3 hours.

24. The method of claim 23, wherein the first incubation period is for about 2 hours to about 3 hours.

25. The method of claim 18, wherein the second incubation period is for less than about 60 minutes.

26. The method of claim 25, wherein the second incubation period is for about 30 minutes to about 45 minutes.

27. The method of claim 18, wherein the detecting the bacteria by detecting the fusion protein includes detecting the fusion protein by visualization with the naked eye, a luminometer, a fluorometer, or a phosphorimeter.

28. The method of claim 18, wherein the detecting the bacteria by detecting the fusion protein has a lower detection limit of 1 CFU/100 mL to 10 CFU/100 mL.

29. The method of claim 18, wherein the bacteriophage is specific to at least one bacterial species or strain of interest.

30. The method of claim 29, wherein the bacteriophage includes a plurality of bacteriophage specific to a plurality of bacterial species or strains of interest.

31. A method of immobilizing bacterial products for detection, the method comprising:
exposing a bacteriophage including an expression construct to bacteria, wherein the expression construct includes a reporter enzyme and a protein affinity tag;
incubating the bacteriophage and the bacteria for an incubation time sufficient for the bacteriophage to infect the bacteria and the bacteria to produce the reporter enzyme and the protein affinity tag as a fusion protein;
immobilizing the fusion protein by binding the protein affinity tag to a complementary substrate; and
detecting the bacteria by detecting the reporter enzyme of the fusion protein.

32. The method of claim 31, wherein the reporter enzyme includes alkaline phosphatase, β-galactosidase, β-glucuronidase, green fluorescent protein, luciferase, neuraminidase, or a derivative of any of the foregoing.

33. The method of claim 31, wherein the protein affinity tag includes a carbohydrate binding module, a chitin binding protein, glutathione-S-transferase, a His tag, a maltose binding protein, or a Strep-tag.

34. The method of claim 31, wherein the protein affinity tag includes a carbohydrate binding module and the complementary substrate is cellulose.

35. The method of claim 31, wherein the incubation time is less than about 60 minutes.

36. The method of claim 35, wherein the incubation time is about 30 minutes to about 45 minutes.

37. The method of claim 31, wherein the detecting the bacteria by detecting the reporter enzyme of the fusion protein includes detecting the reporter enzyme by visualization with the naked eye, a luminometer, a fluorometer, or a phosphorimeter.

38. The method of claim 31, wherein the detecting has a lower detection limit of 1 CFU/100 mL to 10 CFU/100 mL.

39. An expression construct, comprising:
a phage-specific promoter;
a reporter enzyme gene operably linked to the promoter, the reporter enzyme gene selected from alkaline phosphatase, β-galactosidase, β-glucuronidase, green fluorescent protein, luciferase, neuraminidase, and a derivative of any of the foregoing; and
at least a portion of a protein affinity tag gene downstream of the reporter enzyme gene and operably linked to the promoter, the protein affinity tag gene selected from a carbohydrate binding module, a chitin binding protein, glutathione-S-transferase, a His tag, a maltose binding protein, and a Strep-tag.

40. The expression construct of claim 39, further comprising a ribosome binding site and a periplasm-directing leader sequence.

41. A polynucleotide encoding a detectable fusion protein, wherein the polynucleotide comprises the nucleotide sequence of SEQ ID NO: 1.

42. An expression construct comprising the polynucleotide of claim 41.
